(19) United States
(12) Patent Application Publication
(10) Pub. No.: US 2010/0016703 A1
(43) Pub. Date: Jan. 21, 2010

(54) BIO-ELECTRODE POSSESSING A HYDROPHILIC SKIN-CONTACTING LAYER AND AN ELECTROLYTE SUBSTANCE

(75) Inventors: Izmail Batkin, Ottawa (CA); Riccardo Brun Del Re, Ottawa (CA); Hans Kolpin, White Lake (CA)

Correspondence Address: LUCAS & MERCANTI, LLP, 475 PARK AVENUE SOUTH, 15TH FLOOR, NEW YORK, NY, 10016, US

(73) Assignee: CARDIAC BIO-SYSTEMS INC., Ottawa, ON (CA)

(21) Appl. No.: 12/373,658

(22) PCT Filed: Jul. 13, 2007
(86) PCT No.: PCT/CA2007/001245

§ 371 Date: Jan. 13, 2009

Related U.S. Application Data
(60) Provisional application No. 60/807,229, filed on Jul. 13, 2006.

(51) Int. Cl. A61B 5/04

(52) U.S. Cl. 600/392

(57) ABSTRACT

A bio-electrode for conveying electrical signals to, or from a body is constructed with two components, a pre-laminated member and a re-usable electrode assembly. The pre-laminated member comprises a first substrate layer and a second electrolyte-containing layer. The substrate contacts the body and is constructed of a material that is biocompatible, hydrophilic and inherently electrically semi-conductive or conductive. The electrolyte-containing layer, which does not contact the body, is composed of an adhesive, electrolytic hydrogel. Small amounts of moisture and electrolytes from the hydrogel diffuse into the substrate thereby reducing and stabilizing the substrate's electrical resistance. The pre-laminated member can be used with existing re-usable electrode assemblies.

### FIELD OF THE INVENTION

This invention relates to electrodes for detecting physiologic signals from a living body, and for injecting external electrical signals into a body, including a human body. More particularly, it relates to electrodes having a skin-contacting layer material that has some inherent electrical conductivity and is also hydrophilic, and having an aqueous electrolyte-containing layer that is located within or between the skin-contacting layer and the electrode conducting plate.

### BACKGROUND TO THE INVENTION

### Definitions

A summary of certain terms is provided to reduce some of the potential questions with regard to those terms, as they are used herein. It is to be understood that this summary is provided to assist the reader with understanding how the terms relate to each other, but the summary does not restrict the meaning of the terms. The figures and specification more fully establish the meaning for the terms.

“Conditioned substrate” means the portion of a substrate that has been exposed to or saturated with an electrolyte-containing substance, such as a hydrogel or tapwater, on at least one surface such that moisture and electrolytes are at least partially absorbed into the substrate.

“Inherently Dissipative Polymer” or “IDP” means polymers that are dissipative in the context of static control. Such materials have inherent conductivity comparable to materials such as intrinsic semiconductors. Colloquially IDPs may be called ‘poor’ conductors to contrast them with metals, which are ‘excellent’.

“Inherently Conductive Polymer” or “ICP” means polymers that are conductive in the context of static control. ICP refers to polymers with higher conductivity compared to IDP materials, but which are not necessarily as highly conductive as metals. Colloquially these might be called ‘good,’ ‘fair,’ or ‘moderate’ conductors to contrast them with metals, which are ‘excellent.’

“Reservoir” means an area comprising an aqueous substance, including bio-medical hydrogels, tapwater and salt water. The aqueous substance has suitable electrolytic properties, further described herein.

“Substrate” as it pertains to bio-electrodes or electrodes refers to the lower solid, interface layer of the electrode, intended to contact the body.

“Upper” and “lower” with respect to the relative placement of electrode components denote positions that are respectively furthest and nearest to the surface of the body (i.e. the skin).

Physiologic body signals, such as the heart rate, can be measured with devices comprising electrodes. In addition, electrodes can be used to inject electrical signals into the body, such as for bio-impedence measurements or electrode lead loss detection.

Traditionally, bio-electrodes for physiological signal pickup or for signal injection into a body have been constructed using body-contacting materials or substrates that fall into three general categories:
(1) metallic plates or plastics loaded with particles having conductive properties similar to those of a metal;
(2) electrolytic hydrogels; and
(3) insulators.

Electrodes having a body-contacting substrate that fall into category (1) include monolithic metallic electrodes such as stainless steel plate electrodes, and plastic electrodes in which the plastic is loaded with metallic particles or carbon black. Carbon black is a complex particulate form of carbon that exhibits electron conductivity characteristics similar to that of a metal.

Electrodes with a body-contacting substrate as in category (2) include Ag/AgCl gel electrodes which are typically used for diagnostic Electrocardiography (ECG) and Electro-Encephalography (EEG). Also included are various types of gel-based electrodes used for Transcutaneous Electro-Neural Stimulation (TENS), and other specialized uses. The body-contacting material or substrate in the majority of cases is a hydrogel containing an electrolytic solution, which serves as the medium for the effective flow of current between the electrode and the skin.

Examples of electrodes in category (2), which may be of particular interest because they contain an electrolyte stored in a reservoir, are as follows:

U.S. Pat. No. 3,998,215 describes a sponge-like skin-contacting member which absorbs a significant quantity of electrolytic gel. The patent states that “[a] gel pad has impregnated in a porous matrix or held within a cavity, an electrically conductive hydrogel capable of transferring electrical signals between the human body and an electrode of an electrical sensing device when the hydrogel is in contact with the body surface”. The substrate described in this patent provides a supporting structure wherein the matrix is filled with a gel such that direct contact may be provided between gel and skin. This gel establishes the electrical pathway between the skin and the conducting plate.

U.S. Pat. No. 4,215,696 describes a suction electrode having a hydrogel reservoir, wherein the hydrogel reservoir provides electrical contact between the skin and the electrode terminal means to communicate with an electronic device. The disclosed electrode is claimed as being provided with a skin-contacting “microporous, fluid-permeable membrane” through which the electrolyte is ‘stressingly urged’ i.e. forcibly squeezed, via applied pressure, through the pores in the membrane and into direct contact with the body.

As exemplified in the above examples, the electrodes of the prior art which are provided with a reservoir filled with a hydrogel or an electrolytic fluid that initially overlies the skin-contacting layer, establish electrical conduction between the skin and a conducting plate via channels of electrolyte that penetrate an electrically insulating support matrix to provide current pathways between the electrode and the skin.

The use of hydrogels has drawbacks. Substantially exposing the skin to a hydrogel for a prolonged period of time is undesirable as exposure can cause discomfort, irritation and in some circumstances pain upon removal of the electrode. Even short-term exposure to hydrogels can be a nuisance as the hydrogel needs to be wiped away and the skin cleaned after use.

Other electrodes in category (2) use variants of hydrogels. U.S. Pat. No. 4,125,110 describes an electrode in which the body-contacting layer is technically not a ‘hydrogel.’ This is because the suspended liquids disclosed in this patent are a mixture of glycerin, propylene glycol and water, in which the first two components are dominant in terms of mass. This mixture, along with the other specified components, results in a “ . . . colloidal dispersion of a natural organic hydrophilic polysaccharide and salts in an alcohol as the continuous phase.” This is also described as an adhesive polysaccharide gum containing water and electrolytes. This material is closely related to the ‘solid-gel’ hydrogel electrodes of the prior art because the aqueous electrolytic component constitutes the main conductive pathway and the gel matrix has no inherent conductivity of significance, in absence of the aqueous component.

Electrodes in category (3) are capacitive electrodes. Due to the high-impedance of the substrate, these electrodes require impedance conversion electronics attached to or in communication with the substrate. Such electrodes are disclosed in Canadian Patent Application No. 2,280,996 published on Feb. 26, 1991.

Recently, a fourth category of electrode has been disclosed. This type has been called ‘Skin Impedance Matched Biopotential Electrode.’ Such an electrode is described in PCT Patent Application PCT/CA2003/000426 (PCT Publication No. WO2003/079897). This type of electrode is described as being constructed with a semi-conducting substrate i.e. one which possesses some inherent conductivity. Semi-conducting materials are relatively poor conductors. The use of such materials, as opposed to the use of typical ‘dry’ electrodes of category (1), is disclosed as reducing the noise-generating capacity of the resulting contact potential to skin. As such the electrical contact noise is reduced. Like capacitive-type electrodes, these semi-conducting electrodes generally require on-board impedance conversion electronics to compensate for the high-impedance of the substrate. Furthermore, this prior art does not mention the hydrophilicity characteristics of the substrate material nor does it include provision for an aqueous reservoir.

In principle, electrodes of this fourth category could be constructed by using novel polymer IDP and ICP materials. It is known that electrodes constructed of such electrically semi-conductive (IDP) or electrically conductive (ICP) materials are susceptible to some problems. One problem is that some inherently semi-conducting or partially conducting polymers tend to be hydrophilic and water content within the material affects its resistivity. As a result, it is difficult to ensure that electrode resistance remains stable and constant. Such stability of the resistance is required for optimal signal acquisition relative to the impedance of the sensor. Stability is also required to ensure that electrodes remain balanced or symmetrical with respect to other electrodes in the system, such symmetry being needed for the purpose of common-mode noise cancellation.

For the purpose of signal acquisition, electrodes are often used in groups in order to engender cancellation of certain types of noise. Essentially, the signal from one electrode is subtracted from the signal from another electrode. This removes certain types of background noise such as electrical interference. For this common-mode-rejection to work optimally, it is necessary that the resistance of each electrode in the system is constant and balanced with respect to the other electrodes and with respect to the input impedance of the sensors involved. If the resistance of one electrode changes, the symmetry of the system is affected, thus reducing the system's overall performance and noise-cancellation properties. An optimal electrode system cannot be achieved if the electrodes are subject to unpredictable changes in their resistance during the course of the measurement.

Bearing in mind the deficiencies of the prior art, it would be advantageous to provide an electrode similar to the fourth category but which possesses a means of maintaining substantially consistent moisture content within the electrode so as to limit variations in resistance. As well, it would be advantageous to provide such an electrode while maintaining an ostensibly dry body contacting electrode surface.

### SUMMARY OF THE INVENTION

It is an object of the invention to provide an electrode for detecting physiologic signals from or delivering electrical signals to a body. The electrode comprises a body contacting substrate layer, an electrolyte-containing layer and a conducting member. The electrolyte-containing layer is in contact with the body contacting substrate layer and the conducting member. The conducting member is adapted to deliver the physiologic or electrical signals between the body and an external source, through the electrolyte-containing layer and the body contacting substrate layer.

It is another object of the invention to provide an electrode wherein the substrate layer is comprised of a material that is at least partially conductive.

It is another object of the invention to provide an electrode wherein the body contacting substrate layer has the capacity to absorb electrolyte from the electrolyte-containing layer.

It is another object of the invention to provide an electrode wherein the electrolyte-containing layer overlies at least a portion of the body contacting substrate layer.

It is another object of the invention to provide an electrode wherein the body contacting substrate layer comprises a non-metallic, semi-conducting polymer.

It is another object of the invention to provide an electrode wherein the body contacting substrate layer comprises an inherently dissipative polymer.

It is another object of the invention to provide an electrode wherein the body contacting substrate layer further comprises a thermoplastic polyolefin elastomer.

It is another object of the invention to provide an electrode wherein the electrolyte-containing layer is a biocompatible, aqueous hydrogel.

It is another object of the invention to provide an electrode wherein the body contacting substrate layer is hydrophilic.

It is another object of the invention to provide an electrode wherein the conducting member comprises a conducting plate and active electronic mean and wherein the electrolyte-containing layer is in contact with the conducting plate.

It is a further object of the invention to provide an electrode for detecting physiologic signals from or transmitting electrical signals to a body, comprising a pre-laminated member comprising a body contacting layer and an electrolyte-containing layer and a re-usable electrode assembly in contact with the electrolyte-containing layer. The body contacting layer comprises a hydrophilic material that is at least partially conductive and which is capable of absorbing electrolyte from the electrolyte-containing layer. The electrolyte containing layer provides an electrical connection between the body contacting layer and the re-usable electrode assembly.

It is another object of the invention to provide an electrode wherein the pre-laminated member is disposable.

It is a further object of the invention to provide a pre-laminated member to be used with a re-usable electrode assembly in an electrode for detecting physiologic signals from or transmitting electrical signals to a body. The pre-laminated member comprises a body contacting layer, an electrolyte-containing layer and a removable release liner. The electrolyte-containing layer is in contact with the body contacting layer and the removable release liner. The removable release liner can be removed from the pre-laminated member to expose the electrolyte-containing layer prior to removably adhering the body contacting layer and the electrolyte-containing layer to the re-usable electrode assembly.

It is a further object of the invention to provide a pre-laminated member to be used with a re-usable electrode assembly in an electrode for detecting physiologic signals from or transmitting electrical signals to a body. The pre-laminated member comprises a body contacting layer having a first surface and a second surface and a removable release liner. A portion of the first surface is in contact with an electrolyte-containing substance. The removable release liner is in contact with the first surface and the electrolyte-containing substance. The removable release liner can be removed from the pre-laminated member to expose the electrolyte-containing substance prior to removably adhering the first surface of the body contacting layer and the electrolyte-containing substance to the re-usable electrode assembly.

It is another object of the invention to provide a pre-laminated member wherein the electrolyte-containing layer or substance comprises a biocompatible, aqueous hydrogel.

### BRIEF DESCRIPTION OF THE DRAWINGS

FIG. 1 depicts a cross sectional view of one embodiment of a conducting member which can transmit and/or capture signals in accordance with the invention.

FIG. 2 depicts a cross sectional view of an embodiment of a bi-layer body contacting member of the invention in its form prior to use.

FIG. 3 depicts a cross sectional view of an embodiment of a conditioned layer body contacting member of the invention in its form prior to use

FIG. 4 depicts cross sectional views of embodiments of the assembled electrode of the invention, during use. FIG. 4 a) depicts the body contacting member of FIG. 2 and FIG. 4 b) depicts the body contacting member of FIG. 3.

FIG. 5 depicts a cross sectional view of an alternative configuration of the present invention.

FIG. 6 depicts single-lead ECG traces obtained using two electrodes of the invention on a textile chest strap, wherein FIG. 6 a) depicts a human subject in the seated position and trace FIG. 6 b) depicts the same subject walking briskly. The reproduced traces show measurements taken after at least twenty days of wear by the subject.

FIG. 7 shows the reduction of polymer electrical resistance versus time of exposure to moisture reservoirs, wherein the moisture in FIG. 7 a) is tapwater and in FIG. 7 b) is standard TENS electrolytic gel.

### DETAILED DESCRIPTION

The invention in its general form will first be described, and then its implementation in terms of specific embodiments will be detailed with reference to the drawings following hereafter. These embodiments are intended to demonstrate the principle of the invention, and the manner of its implementation. The invention in its broadest and more specific forms will then be further described and defined, in each of the individual claims that conclude this Specification.

Electrodes of the invention possess a number of characterizing features, two of which include:
(1) a substrate layer constructed of a material that is hydrophilic and that possesses at least some inherent electrically conductivity;
(2) an aqueous electrolytic layer (reservoir) overlying the substrate.

Suitable substrate materials include ICPs and IDPs that are also hydrophilic. Even when dry, such materials display some conductivity. It may be desirable to alloy a suitable conducting or semi-conducting polymer material with a non-conducting polymer such as polypropylene or polyurethane in order to obtain a blend that possesses desirable mechanical properties. Typically, but not necessarily, the type of conductivity of the substrate layer is not metallic, but rather ionic in nature. This is typically desirable because ionic conductivity is similar in nature to the conductivity inside living bodies and because ionic conductivity can be significantly enhanced with small amounts of moisture.

Suitable materials for the aqueous layer in the present invention include any electrolytic gels, such as hydrogels designed for use in conventional electrodes. Advantageously, many such gels are adhesive. This eases the application of the substrate and hydrogel of the invention to a conducting, re-usable electrode plate or member. Some such gels are available in sheet form. Such materials are typically originally designed by their manufacturers to serve as the disposable body-contacting layers for conventional re-usable ECG or TENS electrodes. In their originally intended use, the hydrogel layers are meant to serve as the body-contacting (i.e. ‘substrate’) layer wherein one side of the hydrogel layer is to be affixed to an overlying, solid, metallically conductive electrode and the remaining side of exposed hydrogel is intended to contact the skin.

The present invention discloses the placement of an aqueous layer which is very different than conventional hydrogel-using electrode configurations. The substrate interface layer of the invention is a solid polymer layer and this layer contacts the skin while the hydrogel of the invention overlies the substrate and does not contact the skin. Another aspect of the invention is the hydrophilic property of the substrate material and the associated tendency of this material's electrical resistance to decrease through the absorption of a small amount of moisture or electrolyte. The hydrogel layer used in the current invention helps to ensure that the electrical resistance of the electrode is substantially constant.

In an electrode of the present invention, one function of the hydrogel layer (or other suitable aqueous electrolyte reservoir) is to supply moisture and ions for the solid substrate. In another variant of the present invention, the hydrogel layer may also serve to adhere the substrate to a re-usable electrode. In other words, the bi-layer structure of the invention can be used to replace the single gel layer disclosed in the prior art. Electrodes of the invention can also be used to attach to re-usable active electrodes as in category (3) electrodes, described above, which contain on-board impedance conversion electronics. Such impedance conversion electronics may not necessarily be needed if the impedance of the substrate material when used with a hydrogel in accordance with the invention is low enough to provide a signal that can be captured by the outside electronic system.

In one embodiment of the invention, it may be desirable to use a reusable conducting member with on-board electronics in combination with a disposable skin-contacting interface layer that includes a pre-affixed hydrogel layer. The hydrogel used in such a disposable member is typically adhered to the interface layer on one side, and provided with a release liner on the other side of the hydrogel. Prior to use of the electrode of the invention, the release liner is peeled away, thereby exposing the hydrogel, and the hydrogel layer is adhered to the conducting plate. The adhesive properties of the hydrogel may either be inherent in the composition of the interface bi-layer or may be provided through the application of an additional adhesive ring.

In another embodiment, the hydrogel layer may be applied manually to the substrate or conducting plate prior to adhering those two components. Prior to use, the hydrogel must be exposed to the substrate for sufficient time so that the substrate absorbs at least some moisture and electrolytes from the hydrogel. In other words, the substrate must be “conditioned” with the hydrogel.

In still another embodiment, a conditioned substrate is provided, thereby eliminating the requirement to provide an aqueous layer. The substrate of this embodiment is already conditioned or exposed to an electrolyte solution. The conditioned surface may be covered with a removable release liner to protect and preserve the surface during transport and storage. Prior to use, the release liner is removed. A suitable adhesive may be required to removable attach the conditioned substrate to the conducting plate.

With suitable choice of substrate material, electrodes of the invention present a body-contacting layer that appears solid and dry even when it is saturated with a small amount of moisture and electrolyte from the overlying hydrogel.

Compared to prior-art metallic type electrodes described in category (1), above, the electrodes of the present invention can be incorporated into a system that creates less contact potential noise on the skin. Without being bound to any particular theory, noise reduction may result from the ionic conductivity of the conditioned substrate of the invention being similar in nature to that of the body itself. In the case of an active electrode possessing impedance conversion electronics, noise reduction results from the selection of an active electrode input resistor that is matched appropriately to the conditioned substrate's resistance. One example is matching an input resistor which is approximately twenty times that of the resistance of the conditioned substrate disclosed in the fourth category, above.

Compared to prior art electrodes in which a hydrogel is designed to contact the body, described in category (2), above, the electrodes of the invention are more durable and gentler on the skin because the hydrogel is not exposed and does not physically contact the skin. Electrodes of the current invention greatly reduce the amount of moisture and electrolyte components that can reach the skin and are therefore more suitable for prolonged use on the body.

Compared to prior art electrodes of the insulating and semi-conducting type, described in categories (3) and (4), above, the electrodes of the invention possess both reduced and more stable resistivity and thus provide better signals with higher common-mode rejection and less electrical interference noise. Due to the stabilizing effect of the hydrogel layer, electrodes of the invention display more stable resistivity over time.

One embodiment of the present invention utilizes an interface layer with a substrate comprised of semi-conductive polymer (IDP) mixed into a non-conductive supporting matrix composed of mostly polyolefinic components. For example, a thermoplastic polyolefin elastomer containing an IDP is used as the body-contacting interface layer, in which the IDP is mixed in a polypropylene matrix and further alloyed with a common elastomeric component, such as a thermoplastic elastomer like Santoprene™. The resulting blended material presents a robust, comfortable, non-metallic, biocompatible substrate for contacting the body.

The hydrogel component can be one of any of the commercially available sheet hydrogels designed for ECG applications such as those manufactured by Sekisui Plastics Company of Japan.

According to the present invention in one aspect, an electrode is provided with a body contacting, interface layer made of a material that possesses some inherent electrical conductivity. That is, the material is preferably semi-conductive or partially conductive even when dry. Additionally, the electrode of the present invention is provided with a contiguous reservoir such as an electrolyte-containing gel or aqueous layer that overlies at least a part of one surface of the solid, body contacting, interface layer.

The material of the substrate, preferably an IDP or ICP polymer, may be intrinsically semi-conducting or only partially conducting when dry; but when in contact with the electrolyte-containing or aqueous layer, the hydrophilic property of the substrate material ensures that small amounts of moisture and electrolytes from the aqueous layer diffuse into the substrate, thereby reducing the substrate's resistance and stabilizing its resistance against drying that would otherwise occur in the absence of the aqueous layer. The solid, preferably polymeric material of the substrate forms a barrier that substantially eliminates full contact between the electrolyte or gel and the skin. Only small amounts of moisture and aqueous components from the reservoir are needed to diffuse into the polymer in order to substantially increase and stabilize the conductivity of the polymer.

The substrate material should also be bio-compatible, i.e. non-toxic and able to pass standard tests to confirm that it causes minimal skin contact allergic reactions, skin irritation, or sensitization according to a standardized test such as the ISO 10993.

Examples of suitable substrate materials include polyurethane-based IDPs designed for anti-static tubing. In particular, a thermoplastic polyolefin elastomer containing an IDP is used as the body-contacting substrate interface layer in one embodiment. One example of such a substrate is RTP 2899×108110 NS by RTP Company of Winona, Minn., USA. These materials display resistivity analogous to intrinsic semi-conductors when dry. In addition, when exposed to a moisture reservoir of the invention, the resistivity of the substrate can be reduced by orders of magnitude, such as from 100 Mega-Ohm (MoM) to 500 kilo-Ohm (koM), depending in part on environmental conditions. The conditioned substrate becomes highly stable when exposed to the reservoir over time.

The hydrogel can, in one embodiment, also act as an adhesive during use to adhere the substrate of the invention to a re-usable metallic type electrode assembly or to the metallic contact of an active type electrode with on-board impedance conversion electronics. In another embodiment, a suitable adhesive can be added to adhere the substrate to the electrode assembly.

The substrate can also be “pre-laminated” with hydrogel in contact with a portion of at least one surface of the substrate, which is covered by a removable release liner. In this embodiment, when the release liner is removed, the hydrogel is exposed and the member can be adhered to the conducting plate.

Alternatively, a conditioned substrate is provided wherein the substrate material is exposed to the hydrogel and that surface is covered by removable release liner. In this embodiment, when the release liner is removed, the conditioned substrate surface is exposed and the member can be adhered to the conducting plate.

The removal release liner of these embodiments may have a “peel and stick” characteristic, known in the art, thereby facilitating use of the substrate in the invention.

The present invention requires no use of pressure, heat, or manual application of an electrolytic gel on skin in order to attain the desired level of conductivity. The invention comprises a substrate layer that constitutes a complete barrier to the bulk transport of electrolyte therethrough. In this manner, bulk electrolyte is not exposed to the skin.

The interface layer substrate is not considered to be an “insulator” since it was found to possess some intrinsic electrical conductivity and therefore exhibits some conductivity even in the absence of the electrolyte reservoir. This is achieved in respect of the current invention through the use of a polymer which has been mixed with an ICP or IDP material which has inherent electrical conductivity.

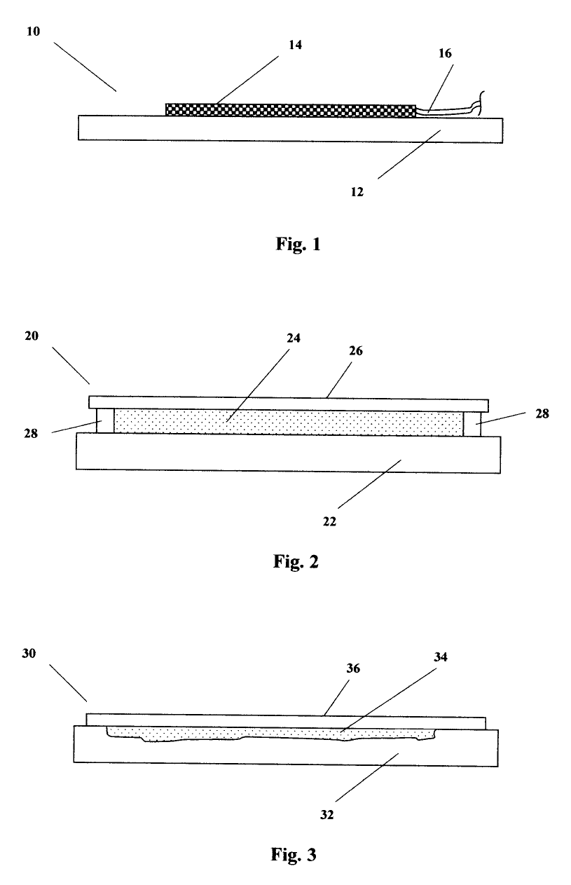

In one embodiment of the invention, the electrode comprises two main components, a re-usable, active electrode assembly 10 as shown in FIG. 1 and a pre-laminated disposable bi-layer 20 as shown in FIG. 2. The electrode assembly 10 comprises a conducting plate 12 and active electronic means 14. The bi-layer component 20 comprises both the substrate 22 and an adhesive hydrogel layer 24 affixed thereto. The hydrogel layer may be adhesive in nature. Optionally, an additional adhesive ring 28 is provided.

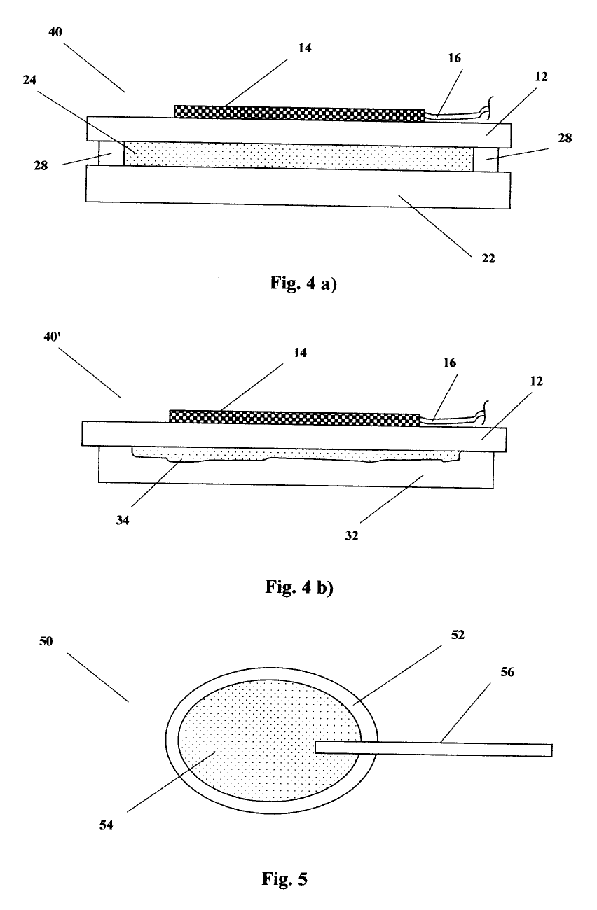

The bi-layer 20 is further provided with a removable release liner 26 that protects and preserves the hydrogel layer 24 during transport and storage. Prior to use, the removable release liner 26 is removed from the hydrogel layer 24, thereby exposing the upper surface of the hydrogel layer 24. The hydrogel layer 24 is then affixed to the conducting plate 12 of the electrode assembly 10 as shown in FIG. 4 a). Assembled, the electrode 40 is now ready to be used.

In another embodiment, the electrode assembly 10 can be used with a conditioned substrate 30 as shown in FIG. 3. The conditioned substrate 30 comprises the substrate 32. Hydrogel is exposed to and in contact with at least one conditioned portion 34 of the substrate 32 such that at least some of the hydrogel is absorbed into the conditioned portion 34 of the substrate 32. A removable release liner 36 protects and preserves the surface of the conditioned portion 34 during transport and storage. Prior to use, the removable release liner 36 is removed from the conditioned portion 34, thereby exposing the upper surface of the conditioned portion 34. The surface of the substrate 32 exposing the conditioned portion 34 is then affixed to the conducting plate 12 of the electrode assembly 10 as shown in FIG. 4 b). Assembled, the electrode 40′ is now ready to be used.

After use, the assembled electrode 40, 40′ may be disassembled for cleaning, storage or reuse. The substrate component 22, 24 (FIG. 4 a) or 32, 34 (FIG. 4 b) is peeled away from the re-usable electrode assembly 10 by separating that component from the conducting plate 12. The substrate component 22, 24 or 32, 34 may be discarded or reused. Infection risk is minimized when the used substrate component is discarded.

Outside connection means 16, as illustrated, such as in the form of a wire, provides a conductive path from the active electronic means 14 and the outside signal interpreting or injecting means (not shown). Such a connection means may also be of any other appropriate form to provide a signal to an outside device. The active electronic means 14 illustrated in FIG. 1 may include a simple impedance conversion circuit or other circuits to modify the input or output of a signal to be provided to the invention.

As the aqueous hydrogel layer 24 or conditioned portion 34 of the invention exerts the greatest moisturizing and electrolytic diffusion influences on the specific area of substrate material to which it is in contact, the invention enables the design of electrodes that have substrates 22, 32 possessing regions of greater or lesser conductivity. If the hydrogel layer 24 has a smaller diameter than the diameter of the solid substrate layer 22, as shown in FIGS. 2 and 4 a), the specific region of the substrate 22 that is in contact with the hydrogel layer 24 is made more conductive than the surrounding substrate regions not in direct contact with the hydrogel layer 24. The electrode in the embodiments illustrated in FIGS. 4 a) and 4 b) comprises a disc-like electrode possessing a substrate 22 with larger diameter compared to the overlying gel 24, also in the form of a disc, located about centrally over the substrate 22. In the illustrated embodiment, the resulting substrate 22 will tend to display lower resistivity near the center and higher resistivity at its periphery. This difference helps to define the signal detection region and may help to minimize detection of signal and noise from the edges of the substrate layer 22.

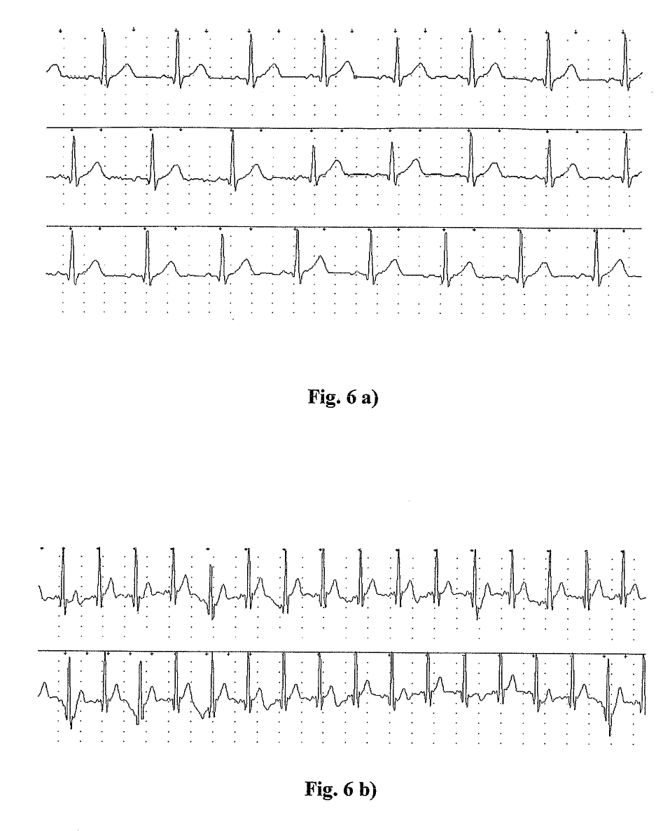

Various configurations of the invention are possible and therefore the shape of the illustrated embodiments is not meant to be restrictive. For example, FIG. 5 depicts an electrode assembly 50 in a somewhat spherical or other irregular shape, which can be used for body measurements in an orifice, such as an ear. A removable release liner is not required in this embodiment since the substrate 52 envelops the hydrogel 54. A portion of the conductor 56 of this embodiment, which may be in the form of a cylinder, is in contact with the hydrogel 54.

Though not shown, an outer shell of material may optionally be provided, such as in the case where it would be desirable for the user to wear the electrode of the invention for extended periods of time or under clothing, to make the electrode substantially “smooth” on the outside. This “shell” may be provided of similar material as the substrate layer, or of any other appropriate material including non-conducting materials. The shell may serve a number of purposes, such as water-proofing of the reusable electrode assembly 10 and ensuring that portions of the electrode do not get caught on the clothing of a user or that the hydrogel layer 24 or conditioned portion 34 does not come into any direct contact with the body or the clothing of the user. Such a shell can enhance the durability of the invention by ensuring that the hydrogel layer 24 or conditioned portion 34 is not exposed for prolonged periods to moisture such that its hydrophilic characteristics are affected. In this manner, a shell would slow down or eliminate the degradation of the electrolyte-containing substance.

It would alternatively be desirable for an electrode of the invention to be provided in a commonly worn item such as a wristwatch which would make it easy to measure an electrophysiological signal over a long period of time while providing a comfortable skin-contacting surface layer. Another application might include a first electrode placed in the ear canal i.e. built into audio headphones such as in-ear-canal headphones and a second electrode on the left side of the user, preferably on the left arm of the user. Such a system could enable detection of the user's heart-rate via a suitably designed personal media or other device which could comprise one or more electrode of the present invention and which could be designed for placement on the user's arm. In this case physiological data such as heart-rate could be accessed immediately via audio feedback or stored in the device for later analysis.

Embodiments of the present invention were tested over various periods of time. In FIG. 6, two single-lead ECG traces are shown. These traces were obtained using two electrodes of the invention on a textile chest strap, worn by a sixty-three year-old male, with a commercially available loop event recorder. In FIG. 6 a) readings were taken while the subject was seated and in FIG. 6 b) readings were taken while the subject walked briskly. The electrodes were worn and measurements were recorded continuously for about one month. The illustrated traces are samples of measurements recorded during the third week. As depicted, the sitting signal is of excellent quality while the walking signal shows acceptable levels of motion artifact. The quality of the traces demonstrate that the electrodes did not suffer degradation due to the long-term exposure to skin. Following testing, the electrodes caused no irritation to the subject's skin.

Several other tests were conducted with the electrodes of the present invention on human subjects, ranging in duration from one minute to one month of contact with the body and continuous measurement. Test data produced excellent quality traces.

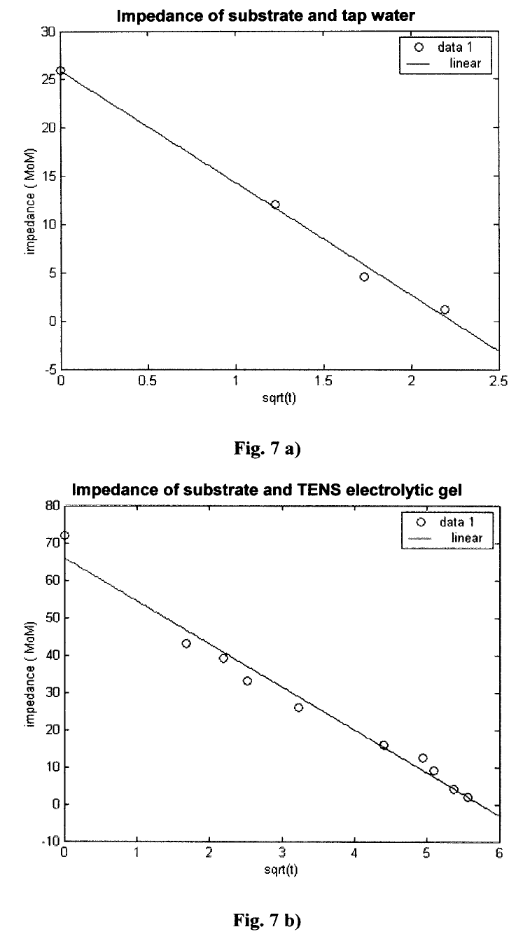

In order to compare various levels of moisture that would typically be encountered during use, flat discs of IDP-containing polymer material were exposed on one surface to various moisture reservoirs. The low-voltage electrical resistance was measured over time using an ordinary digital multi-meter, as depicted in FIGS. 7 a) and 7 b). In this test, the material was RTP 2899×108110 NS. The measured reduction in resistance is due to diffusion of small amounts of moisture and electrolytic ions into the polymer, which was measured to be approximately 6%-8% of polymer dry weight.

The final resistance stabilized at the low value depicted on the graphs, at approximately 1 Mega-Ohm (MoM). A longer exposure to the hydrogel provided a smaller resistance value. At all times, the skin-contacting surface of the material felt dry to the touch, which is consistent with the lack of bulk electrolyte transport through the substrate.

### CONCLUSION

The foregoing has constituted a description of specific embodiments showing how the invention may be applied and put into use. These embodiments are only exemplary. The invention in its broadest, and more specific aspects is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure that has been provided herein.

The embodiments of the invention in which an exclusive property or privilege is claimed are defined as follows:

1. An electrode for detecting physiologic signals from or delivering electrical signals to a body, comprising:
a) a body contacting substrate layer;
b) an electrolyte-containing layer; and
c) a conducting member, said electrolyte-containing layer being in contact with said body contacting substrate layer and said conducting member;
wherein said conducting member is adapted to deliver said signals between said body and an external source, through said electrolyte-containing layer and said body contacting substrate layer.

2. The electrode of claim 1 wherein said body contacting substrate layer is comprised of a material that is at least partially conductive.

3. The electrode of claim 1 wherein said body contacting substrate layer has the capacity to absorb electrolyte from said electrolyte-containing layer.

4. The electrode of claim 1 wherein said electrolyte-containing layer overlies at least a portion of said body contacting substrate layer.

5. The electrode of claim 1 wherein said body contacting substrate layer comprises a non-metallic, semi-conducting polymer.

6. The electrode of claim 5 wherein said body contacting substrate layer comprises an inherently dissipative polymer.

7. The electrode of claim 6 wherein said body contacting substrate layer further comprises a thermoplastic polyolefin elastomer.

8. The electrode of claim 1 wherein said electrolyte-containing layer is a biocompatible, aqueous hydrogel.

9. The electrode of claim 1 wherein said body contacting substrate layer is hydrophilic.

10. The electrode of claim 1 wherein said conducting member comprises a conducting plate and active electronic mean and wherein said electrolyte-containing layer is in contact with said conducting plate.

11. An electrode for detecting physiologic signals from or transmitting electrical signals to a body, comprising:
a) a pre-laminated member comprising a body contacting layer and an electrolyte-containing layer; and
b) a re-usable electrode assembly in contact with said electrolyte-containing layer;
wherein said body contacting layer comprises a hydrophilic material that is at least partially conductive and which is capable of absorbing electrolyte from said electrolyte-containing layer, and wherein the electrolyte containing layer provides an electrical connection between said body contacting layer and said re-usable electrode assembly.

12. The electrode of claim 11 wherein said pre-laminated member is disposable.

13. An electrode for detecting physiologic signals from or delivering electrical signals to a body, comprising:
a) a body contacting substrate layer having a first surface and a second surface; said second surface being in contact with said body; and
b) a conducting member adapted to deliver said signals between said body and an external source; said conducting member being in contact with said first surface;
wherein a portion of said first surface and said conducting member is in contact with an electrolyte-containing substance.

14. A pre-laminated member for use with a re-usable electrode assembly in an electrode for detecting physiologic signals from or transmitting electrical signals to a body, wherein said pre-laminated member comprises:
a) a body contacting layer;
b) an electrolyte-containing layer; and
c) a removable release liner, said electrolyte-containing layer being in contact with said body contacting layer and said removable release liner;
wherein said removable release liner can be removed from said pre-laminated member to expose said electrolyte-containing layer prior to removably adhering said body contacting layer and said electrolyte-containing layer to said re-usable electrode assembly.

15. The pre-laminated member of claim 14 wherein said electrolyte-containing layer comprises a biocompatible, aqueous hydrogel.

16. A pre-laminated member for use with a re-usable electrode assembly in an electrode for detecting physiologic signals from or transmitting electrical signals to a body, wherein said pre-laminated member comprises:
a) a body contacting layer having a first surface and a second surface, wherein a portion of said first surface is in contact with an electrolyte-containing substance; and
b) a removable release liner in contact with said first surface and said electrolyte-containing substance;
wherein said removable release liner can be removed from said pre-laminated member to expose said electrolyte-containing substance prior to removably adhering said first surface to said re-usable electrode assembly.

17. The pre-laminated member of claim 16 wherein said electrolyte-containing substance comprises a biocompatible, aqueous hydrogel.
